(19) United States
(12) Patent Application Publication
(10) Pub. No.: US 2006/0217316 A1
(43) Pub. Date: Sep. 28, 2006

(54) Method for the treatment of prostate cancer

(76) Inventor: Ragab El-Rashidy, Deerfield, IL (US)

Correspondence Address: OLSON & HIERL, LTD., 20 NORTH WACKER DRIVE, 36TH FLOOR, CHICAGO, IL, 60606, US

(21) Appl. No.: 10/551,375

(22) PCT Filed: Mar. 29, 2004
(86) PCT No.: PCT/US04/09485

§ 371 Date: Sep. 29, 2005

Related U.S. Application Data
(60) Provisional application No. 60/459,683, filed on Apr. 2, 2003.

(51) Int. Cl. A61K 38/08; A61K 31/375

(52) U.S. Cl. 514/16; 514/474

(57) ABSTRACT

A method for the treatment of advanced prostate cancer comprises administering to a patient suffering from advanced prostate cancer an androgen suppressing amount of a luteinizing hormone releasing hormone agonist analog and an amount of calcitriol sufficient to enhance the effectiveness of the luteinizing hormone releasing hormone agonist analog against the cancer relative to treatment with the luteinizing hormone releasing hormone agonist analog alone. Preferably the calcitriol is in the form of a stabilized, injectable solution of calcitriol in isotonic saline containing about 1 to about 30 milligrams per milliliter of calcitriol and a sufficient quantity of nonionic surfactant to solubilize the calcitriol therein. Preferably the a luteinizing hormone releasing hormone agonist analog is a nonapeptide or decapeptide agonist, such as leuprolide, goserelin or salts thereof. The method of the present invention affords a surprisingly improved efficacy for treatment of advanced prostate cancer such as androgen-independent prostate cancer (AIPC) or hormone refractory prostate cancer (HRPC) in comparison to treatment with a luteinizing hormone releasing hormone agonist analog alone.

### FIELD OF THE INVENTION

The invention relates to a method for the treatment of prostate cancer. More particularly, this invention relates to a method for the treatment of prostate cancer utilizing 1α,25-dihydroxycholecalciferol (calcitriol) in combination with a luteinizing hormone releasing hormone agonist analog.

### BACKGROUND OF THE INVENTION

Adenocarcinoma of the prostate gland is the most commonly diagnosed malignancy in American men. Although prostate carcinoma is usually a slow growing malignancy, this disease caused considerable mortality. Since prostate cancer rate increases with advancing age, this disease will become an even greater problem as life expectancy increases. Androgens, such as testosterone, regulate the growth, differentiation, and rate of apoptosis in the prostate and its malignancies. Typical therapies for advanced-stage prostate cancer involve androgen withdrawal combined with an androgen receptor antagonist. Such treatments often result in initial tumor regression, but does little to alter the ultimate course of the disease, since androgen-independent tumor progression (also known as hormone refractory prostate cancer) generally ensues within an average of about 17 months. The average survival duration for patients with metastatic prostate cancer is about 2.5 to about 3.5 years. Nearly all patients who die of prostate cancer die from the hormone refractory form of the disease. Currently there is no treatment that is effective in producing prolonged survival in patients with hormone refractory prostate cancer.

Calcitriol (1α,25-dihydroxycholecalciferol) is a biologically active form of vitamin D₃. Calcitriol is important in intestinal calcium transport and bone calcium resorption. An injectable solution containing about 1 to 2 micrograms per milliliter of calcitriol has been used as a treatment for abnormal serum calcium levels. Peehl et al., Journal of Urology, 2002; 168:1583-1588 (herein after “Peehl et al.”), have reported that calcitriol is effective at inhibiting prostate cancer cell growth. Pheel et al. also reported that calcitriol is particularly effective in conjunction with the drug ketoconazole for the treatment of prostate cancer. Oral calcitriol has also been studied in conjunction with docetaxel for the treatment of androgen-independent prostate cancer. See Beer et al. Journal of Clinical Oncology, 2003; 21(1):123-128.

Luteinizing hormone releasing hormone (LHRH agonist analogs are know to be effective in the treatment of prostate cancer. For example, the synthetic LHRH agonist analogs leuprolide and goserelin are potent inhibitors of gonadotropin secretion. Inhibition of gonadotropin results in an inhibition of testosterone production by the testes, and is beneficial in the treatment of prostate cancer.

There is an ongoing need for improved treatments for advanced prostatic cancer such as androgen-independent prostate cancer. The method of the present invention fulfills this need.

### DETAILED DESCRIPTION OF THE INVENTION

A method for the treatment of advanced prostate cancer (APC), such as metastatic androgen-independent prostate cancer comprises administering to a patient having APC an androgen suppressing amount of a luteinizing hormone releasing hormone (LHRH) agonist analog and an amount of calcitriol sufficient to enhance the effectiveness of the LHRH agonist analog against the cancer relative to treatment with the LHRH agonist analog alone. Preferably, the calcitriol and LHRH agonist analog are administered parenterally as separate injections.

Preferably the LHRH agonist analog is a nonapeptide or decapeptide having the structure (I):

(I) 5-oxo-L-Pro-L-His-L-Trp-L-Ser-L-Tyr-Xaa-L-Leu-L-Arg-Yaa (SEQ ID NO: 1), wherein Xaa is a D-amino acid residue or a modified D-amino acid residue; and Yaa is a modified proline residue, such as N-ethyl-L-prolinamide and the like; or a dipeptide comprising a proline and a modified glycine residue, such as L-prolylcarbazamide (Pro-Azgly-NH₂), L-prolylglycinamide (Pro-Gly-CONH₂), and the like.

Preferably, Xaa is a residue selected from the group consisting of O-t-butyl-D-Ser, D-Leu, D-Trp, 2-methyl-D-Trp, N-benzyl-D-His, and 3-(2-naphthyl)-D-Ala. Preferably, Yaa is a residue selected from the group consisting of N-ethyl-L-prolinamide, L-prolylcarbazamide, L-prolylglycinamide, and N-ethylprolylglycinamide.

Suitable LHRH agonist analogs include leuprolide, goserelin, triptorelin, meterelin, buserelin, histrelin, and nafarelin, and salts thereof, which are described in U.S. Pat. No. 6,337,318 to Trigg et al., the relevant disclosures of which are incorporated herein by reference. Preferred LHRH agonist analogs for use in the present invention include leuprolide, goserelin, and salts thereof, such as acetate salts.

Leuprolide is a nonapeptide LHRH agonist analog having the chemical structure (I) wherein Xaa is a D-leucine residue and Yaa is an N-ethyl-L-prolinamide residue. See e.g., U.S. Pat. Nos. 4,005,063, 4,005,194, 4,652,441, 4,677,191, and 5,716,640, which are incorporated herein by reference.

Goserelin is a decapeptide LHRH agonist analog of structure (I) in which Xaa is an O-tert-butyl-D-serine residue and Yaa is a L-prolyl-carbazamide residue (Pro-Azgly) residue.

Triptorelin is a decapeptide LHRH agonist analog of structure (I) in which Xaa is a D-tryptophane residue and Yaa is a L-prolylglycinamide residue (Pro-Gly-CONH₂) residue.

Meterelin is a nonapeptide LHRH agonist analog of structure (I) in which Xaa is a 2-methyl-D-tryptophane residue and Yaa is an N-ethyl-L-prolinamide residue.

Buserelin is a nonapeptide LHRH agonist analog of structure (I) in which Xaa is an O-tert-butyl-D-serine residue and Yaa is a N-ethyl-L-prolinamide residue.

Histrelin is a nonapeptide LHRH agonist analog of structure (I) in which Xaa is a N-benzyl-D-histidine residue and Yaa is an N-ethyl-L-prolinamide residue.

Nafarelin is a decapeptide LHRH agonist analog of structure (I) in which Xaa is a 3-(2-naphthyl)-alanine residue and Yaa is an N-ethyl-L-prolylglycinamide residue.

The calcitriol is in the form of an injectable solution and is administered in a dosage of about 0.1 to about 20 micrograms per kilogram per week (mcg/Kg/week), most preferably about 0.5 to about 10 mcg/Kg/week, based on the patient's weight in kilograms. Preferably the calcitriol is administered as a weekly dose. A typical weekly dose is in the range of about 0.1 to about 20 micrograms (mcg) of calcitriol for an adult patient. Alternatively a depot formulation of calcitriol can be used to provide a sustained release of calcitriol over an extended period of time.

An injectable solution of calcitriol preferably comprises about 1 to about 30 mcg/mL of calcitriol in an isotonic saline medium and a sufficient quantity of nonionic surfactant to solubilize the calcitriol therein. A preferred nonionic surfactant is a polysorbitan, such as polysorbate-20. Preferably the polysorbitan is present in the solution in an amount sufficient to solubilize the calcitriol, most preferably in the range of about 5 to about 20 mg/mL.

In a preferred embodiment, the injectable solution of calcitriol also includes about 1 to about 15 mg/mL of ascorbic acid, more preferably about 2 to about 6 mg/mL of ascorbic acid.

The injectable solution can also include about 1 to about 2 mg/mL of ethylenediamine tetraacetic acid (EDTA) or a salt thereof, such as a sodium salt.

In a particularly preferred embodiment, the injectable solution of calcitriol includes about 5 to about 30 mcg/mL of calcitriol, about 1 to about 15 mg/mL of ascorbic acid, and about 1 to about 2 mg/mL of ethylenediamine tetraacetic acid or a salt thereof, in an isotonic saline medium; and a sufficient quantity of nonionic surfactant to solubilize the calcitiriol therein.

The LHRH agonist analog is preferably administered in a manner conventional for the particular analog in the treatment of prostate concer.

Leuprolide, when utilized is administered preferably as an injectable solution of leuprolide acetate or an injectable depot formulation (i.e., sustained release subcutaneous or intramuscular preparation) of leuprolide (free peptide form) in a physiologically acceptable carrier therefor. Leuprolide acetate is commercially available as an injectable solution in isotonic saline. Leuprolide also can be utilized as depot formulations for sustained-release, and can be administered subcutaneously or intramuscularly. When leuprolide acetate is administered as a solution, the dosage is preferably about 1 mg of leuprolide acetate per day, subcutaneously, for a typical adult patient.

Leuprolide (free peptide) is commercially available in sustained-release depot formulations typically comprising, for example, leuprolide, gelatin, lactic acid/glycolic acid copolymers, D-mannitol, as microspheres, which are reconstituted by dilution with a solution of sodium carboxymethylcellulose, D-mannitol and polysorbate-80 in sterile USP water. Depot formulations of leuprolide are available in a unit doses in the range of about 3.75 mg to 30 mg of leuprolide, for example, as a 3.75 mg weekly depot formulation, a 7.5 mg weekly or monthly depot formulation, a 11.25 mg three-month depot formulation, a 22.5 mg three-month depot formulation, and a 30 mg four-month depot formulation.

When a commercially available 7.5 mg depot formulation of leuprolide is utilized, the dosage is preferably about 7.5 mg of leuprolide, administered by as a single intramuscular injection on a weekly or monthly basis. Alternatively, when leuprolide is administered as a 11.25 mg depot formulation the patient receives about 11.25 mg of the depot formulation as a single intramuscular injection every 3 months; when leuprolide is administered as a 22.5 mg depot formulation the patient receives about 22.5 mg of the depot formulation as a single intramuscular injection every 3 months; or when leuprolide is administered as a 30 mg depot formulation the patient receives about 30 mg of the depot formulation as a single intramuscular injection every 4 months. Leuprolide can also be delivered in the form of an implantable sustained release formulation, for example, as a subcutaneous implant delivering a total dose of about 65 mg of leuprolide over a one year period.

Sustained release formulations of leuprolide are available from TAP Pharmeceuticals Products, Inc., Lake Forest, Ill. under the trade name LUPRON DEPOT®, and from Sanofi Synthelabo, Inc., Malvern, Pa. under the tradename ELEGARD™. An implantable, one-year sustained release formulation of leuprolide acetate (about 65 mg of leuprolide free base per unit dose) is available from Bayer Corp., Pharmeceutical Division, West Haven, Conn., under the trade name VIADUR®.

Goserelin is commercially available as the acetate salt. Goserelin acetate is available from AstraZenica Pharmaceuticals LP, under the tradename ZOLADEX®. ZOLADEX is available as 3.6 mg one-month, and 10.8 mg 3-month sustained release, subcutaneously implantable formulations of goserelin acetate in a biodegradable D,L-lactic acid-glycolic acid copolymer matrix.

As used herein, the term “enhanced effectiveness” and grammatical variations thereof, in relation to the effectiveness of leuprolide as a prostate cancer treatment includes such effects as enhancement of androgen suppressing activity, diminution of side effects, enhanced patient survivability over time, reduced androgen-independent prostate tumor growth, and like effects that improve the clinical utility of LHRH agonist analogs or the quality of life of the patient.

The method of the present invention comprises administering a LHRH agonist analog in conjunction with calcitriol and affords a surprisingly improved efficacy for treatment of advanced prostate cancer such as androgen-independent prostate cancer in comparison with LHRH agonist analog treatment alone. The method of the invention prolongs and enhances the effectiveness of LHRH agonist analogs as a treatment for advanced prostate cancer.

While parenteral administration of calcitriol is preferred, other dosage forms and routes of administration can also be utilized when practicing the present invention. Illustrative such other dosage forms are tablets (oral, sublingual, or buccal), capsules, and the like for oral administration, transdermal patches for percutaneous administration, solutions and suspensions for intranasal administration, suppositories, and the like.

I claim:

1. A method of treating advanced prostate cancer comprising administering to a patient having advanced prostate cancer an androgen suppressing amount of a luteinizing hormone releasing hormone agonist analog and an amount of calcitriol sufficient to enhance the effectiveness of luteinizing hormone releasing hormone agonist analog against the advanced prostate cancer relative to treatment with luteinizing hormone releasing hormone agonist analog alone.

2. The method of claim 1 wherein the luteinizing hormone releasing hormone agonist analog is a nonapeptide or decapeptide having the structure: 5-oxo-L-Pro-L-His-L-Trp-L-Ser-L-Tyr-Xaa-L-Leu-L-Arg-Yaa (SEQ ID NO:1), wherein Xaa is a D-amino acid residue or a modified D-amino acid residue; and Yaa is a modified proline residue or a dipeptide comprising a proline residue and a modified glycine residue.

3. The method of claim 2 wherein Xaa is a residue selected from the group consisting of O-t-butyl-D-Ser, D-Leu, D-Trp, 2-methyl-D-Trp, N-benzyl-D-His, and 3-(2-naphthyl)-D-Ala.

4. The method of claim 2 wherein Yaa is a residue selected from the group consisting of N-ethyl-L-prolinamide, L-prolylcarbazamide, L-prolylglycinamide, and N-ethylprolylglycinamide.

5. The method of claim 1 wherein the calcitriol is in the form of an injectable solution comprising about 1 to about 30 micrograms per milliliter of calcitriol in an isotonic saline medium and a sufficient quantity of nonionic surfactant to solubilize the calcitiriol therein.

6. The method of claim 5 wherein the nonionic surfactant is a polysorbitan.

7. The method of claim 6 wherein the quantity of the polysorbitan is in the range of about 1 to about 10 milligrams per milliliter.

8. The method of claim 5 wherein the injectable solution further comprises about 1 to about 15 milligrams per milliliter of ascorbic acid.

9. The method of claim 5 wherein the injectable solution further comprises about 2 to about 6 milligrams per milliliter of ascorbic acid.

10. The method of claim 5 wherein the injectable solution further comprises about 1 to about 2 milligrams per milliliter of ethylenediamine tetraacetic acid or a salt thereof.

11. The method of claim 5 wherein the injectable solution further comprises about 5 to about 30 micrograms per milliliter of calcitriol, about 1 to about 15 milligrams per milliliter of ascorbic acid, and about 1 to about 2 milligrams per milliliter of ethylenediamine tetraacetic acid or a salt thereof, in an isotonic saline medium; and a sufficient quantity of nonionic surfactant to solubilize the calcitiriol therein.

12. The method of claim 11 wherein the nonionic surfactant is a polysorbitan.

13. The method of claim 12 wherein the quantity of the polysorbitan is in the range of about 1 to about 10 milligrams per milliliter.

14. The method of claim 1 wherein the calcitriol is administered weekly at a dosage in the range of about 0.1 to about 20 micrograms per kilogram, based on the weight of the patient in kilograms.

15. The method of claim 1 wherein the calcitriol is administered weekly at a dosage in the range of about 0.5 to about 10 micrograms per kilogram, based on the weight of the patient in kilograms.

16. The method of claim 1 wherein the luteinizing hormone releasing hormone agonist analog comprises leuprolide, goserelin, or a salt thereof.

17. The method of claim 16 wherein the leuprolide is administered subcutaneously as a daily injection of solution of leuprolide acetate in an isotonic saline solution at a dosage of about 1 milligram per day.

18. The method of claim 16 wherein the leuprolide, goserelin, or salt thereof is in the form of an injectable, sustained release depot formulation.

19. The method of claim 18 wherein the leuprolide is administered a single intramuscular injection per week of about 7.5 mg of leuprolide in the form of a sustained-release depot formulation.

20. The method of claim 18 wherein the leuprolide is administered a single intramuscular injection per month of about 7.5 mg of leuprolide in the form of a sustained-release depot formulation.

21. The method of claim 18 wherein the leuprolide is administered as a single intramuscular injection every three months of about 11.75 mg of leuprolide in the form of a three-month, sustained-release depot formulation.

22. The method of claim 18 wherein the leuprolide is administered as a single intramuscular injection every four months of about 30 mg of leuprolide in the form of a four-month, sustained-release depot formulation.

23. The method of claim 18 wherein the leuprolide is administered subcutaneously in the form of a one-year, sustained release implant containing about 65 mg leuprolide per implant.

24. The method of claim 18 wherein the goserelin is administered subcutaneously in the form of a one-month sustained release implant containing about 3.6 mg of goserelin per implant

24. The method of claim 18 wherein the goserelin is administered subcutaneously in the form of a three-month sustained release implant containing about 10.8 mg of goserelin per implant.
